(19) United States
(12) Patent Application Publication
(10) Pub. No.: US 2009/0136916 A1
(43) Pub. Date: May 28, 2009

(54) METHODS AND MICROARRAYS FOR DETECTING ENTERIC VIRUSES

(75) Inventors: David W. Brown, North Brookfield, MA (US); John E. Herrmann, Northboro, MA (US); Saul Tzipori, Shrewsbury, MA (US); Kerry B. Gunning, Coralville, IA (US)

Correspondence Address: MICHAEL BEST & FRIEDRICH LLP, ONE SOUTH PINCKNEY STREET, P O BOX 1806, MADISON, WI, 53701, US

(73) Assignee: TRUSTEES OF TUFTS COLLEGE, Medford, MA (US)

(21) Appl. No.: 12/191,132

(22) Filed: Aug. 13, 2008

Related U.S. Application Data
(60) Provisional application No. 60/955,461, filed on Aug. 13, 2007.

(51) Int. Cl. C12Q 1/70; C40B 60/10; C07H 21/02; C07H 21/04; C40B 50/14

(52) U.S. Cl. 435/5; 506/38; 536/23.72; 536/24.32; 506/30

(57) ABSTRACT

The present invention relates to methods, microarrays and kits for detecting one or more human astrovirus serotypes in a sample (e.g., a fecal sample) from an individual. The method includes amplifying nucleic acid molecules of the sample with one or more primers, to thereby obtain an amplified nucleic acid product; contacting the amplified nucleic acid product with one or more serotype specific probes having a nucleic acid sequence that is specific for only one astrovirus serotype in the group of astroviruses being assessed, wherein the nucleic acid sequence includes between about 9 and 25 nucleic acid bases (e.g., SEQ ID NO: 5-24); and detecting the hybridization complex. The presence of hybridization complexes with a serotype specific probe indicates the presence of one or more specific astrovirus serotypes, and the absence of hybridization complexes with a serotype specific probe indicates the absence of the specific astrovirus serotype. Identification of the astrovirus serotypes allows for one to diagnose an individual infected with the serotype. The present invention further includes microarrays having any one of the astrovirus specific probe, or kits having microarrays and reagents for carrying out the assay.

### CROSS-REFERENCE TO RELATED APPLICATIONS

This application claims the benefit of U.S. Provisional Application No. 60/955,461, filed Aug. 13, 2007.

### STATEMENT REGARDING FEDERALLY SPONSORED RESEARCH AND DEVELOPMENT

This invention was made with government support under grant N01 AI30050 awarded by National Institutes of Health. The Government has certain rights in the invention.

### BACKGROUND OF THE INVENTION

Human astroviruses are common enteric viruses, and can cause gastrointestinal illness, particularly in children. Human astroviruses are a group of viruses that include specific serotypes, e.g., astrovirus 1, astrovirus 2, astrovirus 3, astrovirus 4, astrovirus 5, astrovirus 6, astrovirus 7, and astrovirus 8. Assays for distinguishing between the astrovirus serotypes either do not exist, or are inefficient and/or time consuming to perform. Therefore, diagnosing astrovirsus infection is further complicated because symptoms of gastrointestinal illness associated with astrovirus (i.e., abdominal pain, vomiting, diarrhea, dehydration), are shared with other diseases or conditions unrelated to astroviruses.

Detection of enteric viral genomes in feces presents a particular challenge because of the great amount of genomic material present from the bacterial flora of the GI tract, from cells shed from the lining of the GI tract, and from ingested material. Non-specific amplification techniques many times suffer from a lack of sensitivity due to the amplification of non-target sequences, and are more appropriate for detection of genomic material 1 in fluids such as CSF, serum, water, and possibly respiratory secretions in which the amounts of competing non-target sequences are limited. A single microarray for a comprehensive panel of pathogens coupled with a non-specific amplification technique, although potentially valuable for screening samples such as serum or CSF, is likely to suffer substantially in sensitivity in the presence of great excesses of non-target sequences, as would be present in feces. For most enteric viruses, fecal samples are the best source of virus, since most enteric viral infections remain localized.

There exists a need for an assay that can efficiently determine whether an astrovirus serotype is present in a sample, particularly a fecal sample, taken from an individual. A further need exists to have tools to determine astrovirus serotype in an infected individual.

### SUMMARY OF THE INVENTION

The present invention relates methods of detecting one or more human astrovirus serotypes (e.g., astrovirus 1, astrovirus 2, astrovirus 3, astrovirus 4, astrovirus 5, astrovirus 6, astrovirus 7, astrovirus 8 or combination thereof) in a group of astroviruses in a sample from an individual. The method includes amplifying nucleic acid molecules of the sample with one or more primers that are specific to a conserved region of the astrovirus serotypes being assessed (e.g., with RT-PCR or with asymmetric PCR), to thereby obtain an amplified nucleic acid product. The methods also involve contacting the amplified nucleic acid product with one or more serotype specific probes having a nucleic acid sequence that is specific for only one astrovirus serotype in the group of astroviruses being assessed, wherein the nucleic acid sequence includes between about 9 and 25 nucleic acid bases; and detecting the hybridization complex. The nucleic acid sequences of the probes of the present invention include any one of SEQ ID NO: 5-24; the complement of any one of SEQ ID NO: 5-24; a nucleic acid sequence having between about 40% and about 100% of contiguous nucleotides (e.g., tiled nucleotides) thereof; a nucleic acid sequence having between about 9 and about 25 contiguous nucleotides thereof, and any combination thereof. “Tiled” probe designs are probes that use the sequences of SEQ ID NO: 5-24 but are just shifted 5′ or 3′ by 1 or more nucleotides. The presence of one or more hybridization complexes with a serotype specific probe indicates the presence of one or more specific astrovirus serotypes, and the absence of one or more hybridization complexes with a serotype specific probe indicates the absence of the specific astrovirus serotype in the sample. Amplification of the nucleic acid molecules can be obtained using RT-PCR, or asymmetric PCR. The methods further include contacting the amplified nucleic acid product with one or more conserved sequence probes having a nucleic acid sequence that is specific for a conserved region shared by all astroviruses in the group of astroviruses being assessed. The conserved sequence probes have a nucleic acid sequence of AGAGCAACTCCATCGCAT (SEQ ID NO: 3) or GAGGGGAGGACCAAAGAA (SEQ ID NO: 4); the complement of SEQ ID NO: 3 or 4; a nucleic acid sequence having between about 40% and about 100% of contiguous nucleotides thereof, a nucleic acid sequence having between about 9 and about 25 contiguous nucleotides of thereof; and any combination thereof. The steps of the invention, in one aspect, include incorporating a detectable label into the amplified nucleic acid product from the sample. Primers that are specific to a conserved region of the astrovirus serotypes being assessed and are used to amplify nucleic acid molecules of the sample, in an embodiment, have a nucleic acid sequence of ACTGCCTRTCWCGGACTG (SEQ ID NO: 1) or TGTGACACCYTGTTTCCT (SEQ ID NO: 2). In an embodiment, SEQ ID NO-2 is labeled with Cy-3 at the 5′ end. In an embodiment, the nucleic acid molecules from the sample are reverse transcribed to thereby obtain DNA; and the DNA can be amplified and labeled.

In another embodiment, the nucleic acid molecules of sample are isolated, and then contacted with one or more primers that are specific to a conserved region of the astrovirus serotypes being assessed, wherein one of the primers incorporates a tag into the amplified nucleic acid molecules. These steps result in an amplified nucleic acid product having a labeled nucleic acid strand and an unlabeled nucleic acid strand. The methods involve digesting the unlabeled nucleic acid strand to thereby obtained an amplified labeled nucleic acid product; and contacting the amplified nucleic acid product, as described herein, with the probes of the present invention, and detecting the hybridization complex. The presence of one or more hybridization complexes indicates the presence of one or more species specific astroviruses, and the absence of the complex indicates the absence of a species specific astrovirus.

Methods of the present invention include methods for diagnosing an individual having a disease or condition associated with an astrovirus (e.g., gastroenteritis). The methods involve determining the presence or absence of one or more nucleic acid molecules from a sample from the individual that hybridize to one or more nucleic acid probes of the present invention. The presence, absence, level or percentage of one or more complexes indicates the presence or absence of the disease or condition. Similarly, methods of the present invention also relate to methods for monitoring treatment or efficacy of therapy for an individual having a disease or condition associated with an astrovirus. The steps include determining the presence or absence of one or more nucleic acid molecules from a sample, as described above, at one or more time points; and comparing or analyzing the presence or absence of the one or more complexes at the one or more time points. The comparison or analysis indicates the efficacy of therapy.

The present invention includes an array for the identification of one or more astrovirus serotypes, wherein the array comprises one or more nucleic acid probes of the present invention, as described herein, wherein each molecule is bound to the surface of a solid support in a different localized area. The solid support, in one aspect, can be epoxide, glass, silica chips, nylon membrane, polymer, plastic, ceramic, metal, and optical fiber. The solid support has more than one array (e.g., between about 1 and about 48 different arrays), and can be duplicated 2 or more times. In an embodiment, more than one (e.g., two or three) nucleic acid molecules are used to identify one serotype.

In yet another aspect, kits are an embodiment in the present invention. The kits include one or more arrays for the identification of one or more astrovirus serotypes, as described herein, and one or more reagents used for carrying out a nucleic acid hybridization assay. Examples of such regents include compounds used to detect hybridization; unlabeled primers that are specific to a conserved region of the astrovirus serotypes being assessed, labeled primers that are specific to a conserved region of the astrovirus serotypes being assessed, washing solutions; and buffers.

The present invention further relates to the isolated nucleic acid molecules that identify specific astrovirus serotypes. The molecules or probes have a nucleic acid sequence of any one of SEQ ID NO: 5-24; the complement of any one of SEQ ID NO: 5-24; a nucleic acid sequence having between about 40% and about 100% of contiguous nucleotides thereof; a nucleic acid sequence having between about 9 and about 25 contiguous nucleotides thereof; and any combination thereof. The isolated nucleic acid molecule can be DNA or RNA molecule, or a probe that binds to an astrovirus serotype.

The present invention also includes methods of making an array for the identification of an astrovirus serotype. The methods pertain to attaching to a solid support one or more nucleic acid molecules of the present invention, wherein each molecule is attached to the surface of a solid support in a different localized area.

The nucleic acid molecules are from a solution having a concentration of between about 1 μM and 200 μM. In an example, more than one array (e.g., between about 1 about 48 arrays) is printed on one glass slide, and the same array is duplicated 2 or more times. The methods further include synthesizing said nucleic acid molecule and/or inserting or integrating the probes within the solid support.

The present invention advantageously provides a rapid and reliable assay for determining which astrovirus serotype exists in a sample. This assay can even be performed using a fecal sample, which includes a lot of genomic material. The microarray and methods of the present invention allow one to better diagnose gastrointestinal illness due to an astrovirus, and therefore allows one to better treat the individual.

### BRIEF DESCRIPTION OF THE DRAWINGS

The patent or application file contains at least one drawing executed in color. Copies of this patent or patent application publication with color drawings will be provided by the Office upon request and payment of the necessary fee.

FIG. 1 is a diagram showing the design of a microarray, in one embodiment, of the present invention, and the nucleic acid probes used to identify the specific astrovirus isolate, and the corresponding astrovirus isolates. Locations of the probes are indicated under the figure. A=site 3. B=site 4. C=site 5. c1=common sequence, site 1. c2=common sequence, site 2.

FIGS. 2A-B show an alignment of astrovirus sequences from eight serotypes in the region amplified by the RT-PCR primers used for detection and generation of labeled targets for microarray hybridization. The GenBank accession number for each sequence is listed to the left. In parentheses are the serotype designations. Primers used for RT-PCR are indicated in aqua in the color drawing, and appear as darkly shaded in black and white. Probe sequences at conserved sites (1 and 2) and sites used for type identification (3, 4, and 5) are indicated in yellow in the color drawing, and appear as lightly shaded in black and white. Nucleotides that differ from the consensus are highlighted in green, and appear has having a medium shading in black and white. Astroviruses 2 and 4 are identical at site 3, and astroviruses 1 and 5 are identical at site 4. Probes for these were not included in the microarray.

FIG. 3 is a photograph of oligonucleotide microarrays for distinguishing the eight different types of human astrovirus. RT-PCR was performed using a single pair of primers at equimolar concentrations. The antisense primer was labeled with Cy3. The RT-PCR products were enzymatically digested to remove the unlabeled (and unprotected) sense strands, and the remaining labeled antisense targets were column purified and applied to the microarray consisting of predominantly 17 mer positive sense probes. Duplicate dots in the upper right and lower left of each array are two conserved sequences in common to all the astroviruses. Type specific probes are clustered as two to three pairs of duplicate dots on the array. Locations of probes on the microarray are provided in FIG. 1.

### DETAILED DESCRIPTION OF THE INVENTION

The present invention relates to arrays and methods for identifying one or more human astrovirus serotypes. The present invention pertains to specific nucleic acid molecules that are useful in identifying the specific astrovirus serotype, and diseases and/or conditions related to it. The diagnostic approach of the present invention enables rapid detection and characterization of human astrovirus isolates. The assay can be performed, in an embodiment, using direct labeling of RT-PCR products with a single fluorophore per target molecule without the need for a second target amplification step or enzyme-based signal amplification. In yet another embodiment, enzymatic digestion of the non-labeled strand enables production of labeled ssDNA targets without compromising the optimum primer concentrations for initial detection of the virus, as would occur with asymmetric amplification procedures. Use of conserved primers for the initial RT-PCR improves chances of detecting uncharacterized isolates. By using short nucleotides (17-mers) as probes in the oligonucleotide microarray, single nucleotide changes were detected, thus improving identification of serotypes differing at the sites of the probe sequences. As more isolates (e.g., serotypes) are characterized, the microarray can be expanded to account for greater diversity as such diversity is encountered.

An RT-PCR, a target labeling system, and a microarray of short oligonucleotides for detection and characterization of human astroviruses were designed. Use of short oligonucleotides offers a sensitive means of distinguishing closely related amplicons. Proof of principle was demonstrated with the current array distinguishing eight known serotypes of human astroviruses, as shown in the Exemplification.

The method or array of the present invention is the first of its kind to have an ability to identify specific isolates of astroviruses, especially from a sample having such an extensive amount of genomic material (e.g., fecal sample). Although an embodiment of the invention includes obtaining fecal samples, the methods of the present invention can be performed using any number of samples including samples from the feces, saliva, sputum, aspirate, blood, plasma, cerebrospinal fluid, aspirate, tissue, skin, urine, mucus, etc.

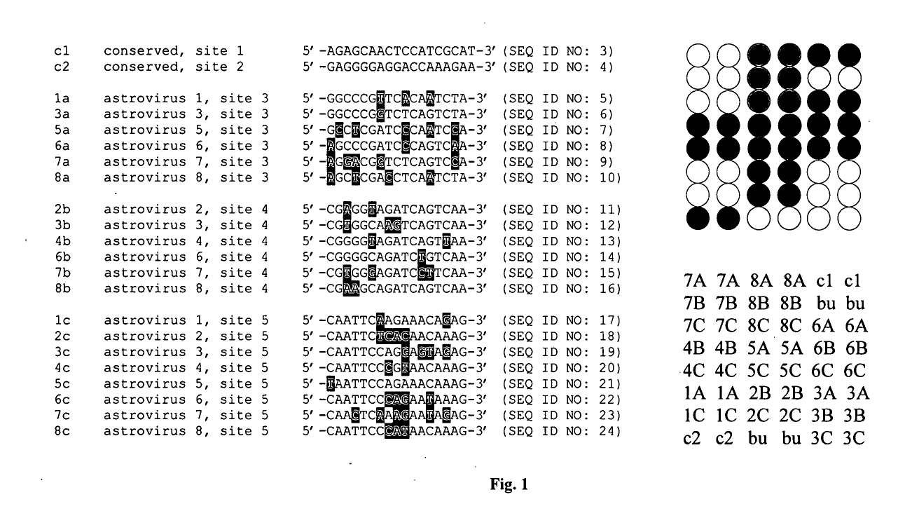

The present invention includes methods for assessing the presence of one or more specific astrovirus serotypes in a sample by assessing the presence or absence of nucleic acid sequences specific for that specific astrovirus serotype. Specifically, the method includes contacting nucleic acid molecules obtained (e.g., amplified and labeled) from a sample with the probes of the present invention. This step occurs under conditions suitable for hybridization to form a complex or hybrid, and the hybrids are detected. The presence of complexes correlate with the specific serotypes listed in FIG. 1.

Such an analysis is helpful in assessing whether the individual from whom the sample is taken has been infected with one or more astroviruses. A diagnosis allows one to more effectively treat diseases associated with the virus. Similarly, ruling out infection also allows one to determine other potential causes of the patient's symptoms. Additionally, treatment can be monitored on a patient to determine if the patient is getting better and ridding the infection from the body.

More specifically, the present invention includes, in part, methods for identifying one or more astrovirus serotypes through the hybridization of the nucleic acid molecules described herein. Astrovirus serotypes refer to designations of specific astroviruses and include e.g., astrovirus 1, astrovirus 2, astrovirus 3, astrovirus 4, astrovirus 5, astrovirus 6, astrovirus 7, and astrovirus 8. Additional serotypes are included in the present invention, and include those later classified, designated, or characterized. In such a case, 9-25 mer (e.g., 17 mer) probes can be designed that are unique to the specific serotype, as done with astroviruses 1-8. See Exemplification. Such probes can be further included in the microarrays and methods of the present invention.

In a preferred embodiment, methods for identifying a nucleic acid sequence involve the use of an array. An “array,” “microarray,” “DNA chip,” “biochip,” or “oligo chip” may be used interchangeably and refers to a grid of spots or droplets of genetic material of known sequences in defined locations or known positions. The advantage of using an array is the ability to test a sample against hundreds of nucleic acid sequences at once. The array of probes can be laid down in rows and columns. As shown in FIG. 1, arrays (8×6 droplets???) are arranged on a support. In an embodiment, the same array is repeated more than once to verify the accuracy of results obtained using the arrays. The actual physical arrangement of probes on the chip is not essential, provided that the spatial location of each probe in an array is known. When the spatial location of each probe is known, the data from the probes can be collected and processed. In processing the data, the hybridization signals from the respective probes can be reasserted into any conceptual array desired for subsequent data reduction whatever the physical arrangement of probes on the chip. The present invention includes arrays having one or more of the nucleic acid molecules described herein (any one of SEQ ID NOs:5-24; the complement thereof, a nucleic acid sequence having between about 40% and about 100% of contiguous nucleotides (e.g., tiled nucleotides) of any one of SEQ ID NO: 5-24; a nucleic acid sequence having between about 9 and about 25 contiguous nucleotides of any one of SEQ ID NO: 5-24; a reverse complement thereof, and any combination thereof) bound or attached thereto.

The present invention encompasses combinations of the nucleic acid molecules described herein arranged in an array. The array can be tailored to identify certain all or some astrovirus serotypes. As such, the present invention includes having nucleic acid molecules that identify one or more of the astrovirus serotypes. For example, the present invention includes an array having at least about 100%, 90%, 80%, 70%, 60%, 50%, 40%, 30% 20% or 10% of the nucleic acid molecules disclosed herein. The present invention also includes having a particular combination of the nucleic acid molecules described herein (e.g., about 1, 2, 3, 4, 5, 6, 7, 8, 9, 10, 11 12, 13, 14, 15, 16, 17, 18, 19, 20, 21, 22, or any combination thereof) arranged in an array format.

The genetic material is systematically arranged on a solid support that includes, e.g., glass, silica chips, nylon (polyamide) membrane, polymer, plastic, ceramic, metal, coated on optical fibers, or infused into gel, matrix. In addition to solid arrays, any format now known or later developed can be used to carry out the steps of the present invention. In one aspect, “liquid array” platforms can also be used to carry out the steps of the present invention. Examples include polystyrene beads (e.g., from Luminex), acid-etched bar-coded fiber optic cable chunk (e.g., from CyVera (formerly Cidra)), gold nanoparticles, transponders, and silicon-based “beads” (e.g., True Materials). The steps of the present invention include in situ synthesis array platforms (e.g., from Affymetrix and Nimblegen).

With respect to solid support arrays, examples of solid support types include slides, plates, chips, dipsticks, or other types known in the art or later developed. The solid support can also be coated to facilitate attachment of the oligonucleotides to the surface of the solid support. Any of a variety of methods known in the art may be used to immobilize oligonucleotides to a solid support. The oligonucleotides can be attached directly to the solid supports by epoxide/amine coupling chemistry. See Eggers et al. Advances in DNA Sequencing Technology, SPIE conference proceedings (1993). Another commonly used method consists of the non-covalent coating of the solid support with avidin or streptavidin and the immobilization of biotinylated oligonucleotide probes. By oligonucleotide probes is meant nucleic acid sequences complementary to a species/serotype-specific target sequence.

Using a solid support having the nucleic acid molecules bound thereto, the method of the present invention involves contacting the nucleic acid molecules described herein with nucleic acid molecules obtained from a sample to be tested under conditions suitable for hybridization with one another. A sample is obtained from the individual to be tested and can consist of feces, saliva, sputum, aspirate, blood, plasma, cerebrospinal fluid, aspirate, tissue, skin, urine, mucus, or cultured organisms grown in vitro. The nucleic acid of the sample can be amplified and labeled so that it is suitable for hybridizing with the nucleic acid molecules of the present invention. The term, “amplifying,” refers to increasing the number of copies of a specific polynucleotide. As it applies to polynucleotide molecules, amplification means the production of multiple copies of a polynucleotide molecule, or a portion of a polynucleotide molecule, typically starting from a small amount of a polynucleotide (e.g., a viral genome), where the amplified material (e.g., a viral PCR amplicon) is typically detectable. In an embodiment, methods involved primers that are specific to a conserved region of the astrovirus serotypes being assessed. The specificity of the primers increases the likelihood that astrovirus nucleic acid molecules will be amplified. Amplification of polynucleotides encompasses a variety of chemical and enzymatic processes. The generation of multiple DNA copies from one or a few copies of a template DNA molecule during a polymerase chain reaction (PCR), a strand displacement amplification (SDA) reaction, a transcription mediated amplification (TMA) reaction, a nucleic acid sequence-based amplification (NASBA) reaction, or a ligase chain reaction (LCR) are forms of amplification. Amplification is not limited to the strict duplication of the starting molecule. For example, the generation of multiple cDNA molecules from a limited amount of viral RNA in a sample using RT-PCR is a form of amplification.

In embodiments of these methods, the step of amplifying the astrovirus serotype genetic material is by reverse transcription (RT) combined with polymerase chain reaction (PCR). This PCR can use a primer pair that is specific to a conserved region of the astrovirus serotypes being assessed, and comprises the nucleotide sequences of, e.g., SEQ ID NOS:1 and 2. Generally, the PCR process consists of introducing a molar excess of two or more extendable oligonucleotide primers to a reaction mixture comprising the desired target sequence(s), where the primers are complementary to opposite strands of the double stranded target sequence. The reaction mixture is subjected to a program of thermal cycling in the presence of a DNA polymerase, resulting in the amplification of the desired target sequence flanked by the DNA primers. Reverse transcriptase PCR (RT-PCR) is a PCR reaction that uses RNA template and a reverse transcriptase, or an enzyme having reverse transcriptase activity, to first generate a single stranded DNA molecule prior to the multiple cycles of DNA-dependent DNA polymerase primer elongation. Methods for a wide variety of PCR applications are widely known in the art, and described in many sources, for example, Ausubel et al. (eds.), Current Protocols in Molecular Biology, Section 15, John Wiley & Sons, Inc., New York (1994). PCR also can be used to detect the existence of the defined sequence in a DNA sample.

In an embodiment, amplification is includes or is optionally followed by additional steps, such as labeling, sequencing, purification, isolation, hybridization, size resolution, expression, detecting and/or cloning.

As used herein, the expression “asymmetric PCR” refers to the preferential PCR amplification of one strand of a DNA target by adjusting the molar concentration of the primers in a primer pair so that they are unequal. An asymmetric PCR reaction produces a predominantly single-stranded product and a smaller quantity of a double-stranded product as a result of the unequal primer concentrations. As asymmetric PCR proceeds, the lower concentration primer is quantitatively incorporated into a double-stranded DNA amplicon, but the higher concentration primer continues to prime DNA synthesis, resulting in continued accumulation of a single stranded product.

Briefly, PCR is performed with the use of a DNA polymerase enzyme and include, for example, one that is isolated from a genetically engineered bacterium, Thermus aquaticus (Taq). Other DNA polymerases include, e.g., ThermalAce™ high fidelity polymerase (Invitrogen), TthI polymerase, VENT polymerase or Pfu polymerase. The polymerase, along with the primers and a supply of the four nucleotide bases (adenine, guanine, cytosine and thymine) is provided. Under certain conditions (e.g., 95° C. for 30 seconds), the DNA is denatured to allow the strands to separate. As the DNA solution cools, the primers bind to the DNA strands, and then the solution is heated to promote the Taq polymerase to take effect. Mullis, K. B. Scientific American 256:56-65 (1990). Other known methods, or methods developed in the future can be used so long as the DNA of the sample is amplified or replicated.

In an embodiment, after a round of RT-PCR with a single pair of primers of low degeneracy, the RT-PCR product is labeled using an anti-sense primer (e.g., with Cy-3) during amplification. Single stranded target DNA is obtained by enzymatic degradation of the unlabeled sense stand followed by column purification of the labeled antisense strand. Single stranded antisense target DNA can also be obtained by asymmetric PCR, described herein, using excess labeled sense primer. In an embodiment, either primer could be labeled to thereby label either strand. Labeling the anti-sense strand allows the sense orientation to be used for the probe designs, whereas labeling the sense-strand allows the anti-sense orientation to be used for the oligo probe design. Conversely, if the oligo probes are designed to use the sense orientation then the labeled primer should be the anti-sense sequence, and visa versa.

Several labels exist to facilitate detection of a nucleic acid molecule complex. Techniques for labeling and labels, that are known in the art or developed in the future, can be used. In a preferred embodiment, the label is simultaneously incorporated during the amplification step in the preparation of the sample nucleic acids. For example, PCR with labeled primers or labeled nucleotides will provide a labeled amplification product. The nucleic acid (e.g., DNA) is amplified in the presence of labeled deoxynucleotide triphosphates (dNTPs). In a preferred embodiment, transcription amplification, as described above, using a labeled nucleotide (e.g., fluorescein-labeled UTP and/or CTP) incorporates a label into the transcribed nucleic acids.

Detectable labels suitable for use in the present invention include any composition detectable by spectroscopic, photochemical, biochemical, immunochemical, electrical, optical or chemical means. The most frequently used labels are fluorochromes like Cy3, Cy5 and Cy7 suitable for analyzing an array by using commercially available array scanners (e.g., Axon, General Scanning, and Genetic Microsystem). Other labels that can be used in the present invention include biotin for staining with labeled streptavidin conjugate, magnetic beads (e.g., Dynabeads®), dendrimers, fluorescent proteins and dyes (e.g., fluorescein, Texas red, rhodamine, green fluorescent protein, and the like, see, e.g., Molecular Probes, Eugene, Oreg., USA), radioactive labels (e.g., ³H, ¹²⁵I, ³⁵S, ¹⁴C, or ³²p), enzymes (e.g., horse radish peroxidase, alkaline phosphatase and others commonly used in an ELISA), and calorimetric labels such as colloidal gold (e.g., gold particles in the 40-80 nm diameter size range scatter green light with high efficiency) or colored glass or plastic (e.g., polystyrene, polypropylene, latex, etc.) beads. Patents teaching the use of such labels include WO 97/27317, and U.S. Pat. Nos. 3,817,837; 3,850,752; 3,939,350; 3,996,345; 4,277,437; 4,275,149; and 4,366,241.

A fluorescent label is preferred because it provides a very strong signal with low background. It is also optically detectable at high resolution and sensitivity through a quick scanning procedure. The nucleic acid samples can all be labeled with a single label, e.g., a single fluorescent label. Alternatively, in another embodiment, different nucleic acid samples can be simultaneously hybridized where each nucleic acid sample has a different label. For instance, one target could have a green fluorescent label and a second target could have a red fluorescent label. The scanning step will distinguish cites of binding of the red label from those binding the green fluorescent label. Each nucleic acid sample (target nucleic acid) can be analyzed independently from one another.

The sample can be purified to remove unincorporated label or dye. Purification allows reduction in the overall slide background (e.g., the inter-spot space area) that would be caused by the “un-used” labeled primer. This would in turn impact the overall sensitivity of the array, effecting the signal-to-noise ratio.

Once the sample is prepared, it can be subjected to the nucleic acid molecules of the present invention for hybridization. Hybridization refers to base pairing between single strands of polynucleotides at least partially complementary to form a double-stranded molecule or a partially double-stranded molecule. With respect to the present invention, the labeled DNA of the sample hybridizes with the oligonucleotides on the solid support. Hybridization conditions include variables such as temperature, time, humidity, buffers and reagents added, salt concentration and washing reagents. Preferably, hybridization occurs at high stringency conditions (e.g., 55° C., for 16 hours, 3×SSC). Examples of stringency conditions are described herein. Methods for hybridization are known, and such methods are provided in U.S. Pat. No. 5,837,490, by Jacobs et al. The solid support can then be washed one or more times with buffers to remove unhybridized nucleic acid molecules. Hybridization forms a complex between the nucleic acid of the present invention and nucleic acid of the sample.

Hybridization assay procedures and conditions will vary depending on the application and are selected in accordance with the general binding methods known including those referred to in: Maniatis et al. Molecular Cloning: A Laboratory Manual (2.sup.nd Ed. Cold Spring Harbor, N.Y., 1989); Berger and Kimmel Methods in Enzymology, Vol. 152, Guide to Molecular Cloning Techniques (Academic Press, Inc., San Diego, Calif., 1987); Young and Davism, P.N.A.S, 80: 1194 (1983). Methods and apparatus for carrying out repeated and controlled hybridization reactions have been described in U.S. Pat. Nos. 5,871,928, 5,874,219, 6,045,996 and 6,386,749, 6,391,623.

The complex, which is labeled, can be detected and quantified. Detection of the array can be performed by autoradiography or in real time to determine the presence of hybridized products at particular locations on the solid support. In particular, detection can occur using scanners that emit light from a laser at specific frequency. In one example, an Affymetrix 428 scanner at an excitation wavelength of 532 nm, an emission wavelength of 570 nm, laser power at 80% and gain at 50% was used. Scanners and other devices, including those known and later developed, for detecting the labeled hybridized complexes can be used. These measurements are converted to electronic signals that can be analyzed. The raw data optionally are filtered and/or normalized. Filtering refers to removing data from the analysis that does not contribute information to the experimental outcome, e.g., does not contribute to the identification of a serotype. Normalizing data refers to, in one embodiment, a linear transformation to correct for variables within the experimental process.

In addition to detecting the presence or absence (e.g., below a detectable threshold), quantification can also occur and be provided in a level or percentage. While in one embodiment, as shown in the Exemplification, presence or absence of hybridization is demonstrated, signal intensity relative to other probes can also be used for quantification. As a general rule, the more hybridization of complexes that is present (e.g., presence of the serotype in the sample), the more intense the probe signal. In an embodiment in which PCR amplification occurs, the intensity does not directly reflect absolute numbers, but rather is proportional to a relative amount in the original sample. Such quantification of hybridization complexes can be carried out using methods known in the art. To achieve quantification, one can develop a standard curve hybridization data set that uses the “common region” probe sequences (e.g., site 1 & 2) and serial dilution of a known serotype to generate hybridization signal data. Then patient samples can be quantified based on their “common region” probe hybridization signal strength.

The data can then be analyzed by a qualified person or computerized system. In an embodiment, the presence of hybridization of the nucleic acid molecules of the present invention correlates to the presence of the corresponding serotype in the sample. One can compare the spot having a detectable hybrid complex, against a table or database containing information about the spots on which the nucleic acid molecules were bound, and with which particular serotype they correlate. FIG. 1 has a table that lists the astrovirus serotypes and the sequence of the probe to which they correlate. After such a comparison, the serotype can be identified in the sample. One or more nucleic acid molecules can correlate to a particular serotype. In some embodiments, at least 2 probes correlate to or identify an astrovirus serotype. Having more than one occurrence of hybridization with more than one probe can, in some embodiments, provide for a more accurate identification.

Additionally, the microarray of the present invention includes two probes, SEQ ID NOS: 3 and 4, that bind to the conserved region of the group of astroviruses being tested. In one aspect, hybridization with these probes can be used as a control. If a serotype of the astrovirus is present (e.g., if there is serotype-specific binding), then there should also be binding with the conserved probes as well. In the case in which serotype specific hybridization occurs, and no hybridization with the conserved probes occurs, then results indicates that there may be aberrant isolate. If the opposite occurs, then there may be an indication that a new astroviral serotype exists, one not yet characterized, but shares the same conserved region as the other serotypes in the group of astroviruses.

The presence of hybridization, as detected in some embodiments by fluorescence, is compared to controls (e.g., positive and/or negative controls).

In one embodiment, a positive control can be used (e.g., a sample containing all astrovirus serotypes being assayed. Negative controls can also be used. Negative controls, in an embodiment, include nucleic acid not found in the astroviruses being tested, or no nucleic acid. The “non-astrovirus nucleic acid” negative control aids in help demonstrating specificity of the probe set and conditions to astrovirus targets while the “no nucleic acid” negative control assists in determining overall slide background (e.g., probe spot background vs. slide (or inter spot space) background).

The methods of the present invention also involve determining the level or percentage of a particular serotype in a sample. Data can be generated for mean detection levels or percentage of known quantities of a serotype and can be used to compare a sample of unknown quantity to determine the level or percentage of the serotype in the sample. In one embodiment, threshold levels or percentages (e.g., low, medium and high) of serotypes can be established using known quantities of serotypes, and compared to an unknown level or percentages of serotypes in a sample. Detection of one or more serotypes above the high threshold level signifies high quantities of the particular serotypes, detection of a medium threshold level indicates a mid-level quantity of the serotypes in the sample, and detection of serotypes below the low threshold levels indicate low quantities of the serotypes in the sample.

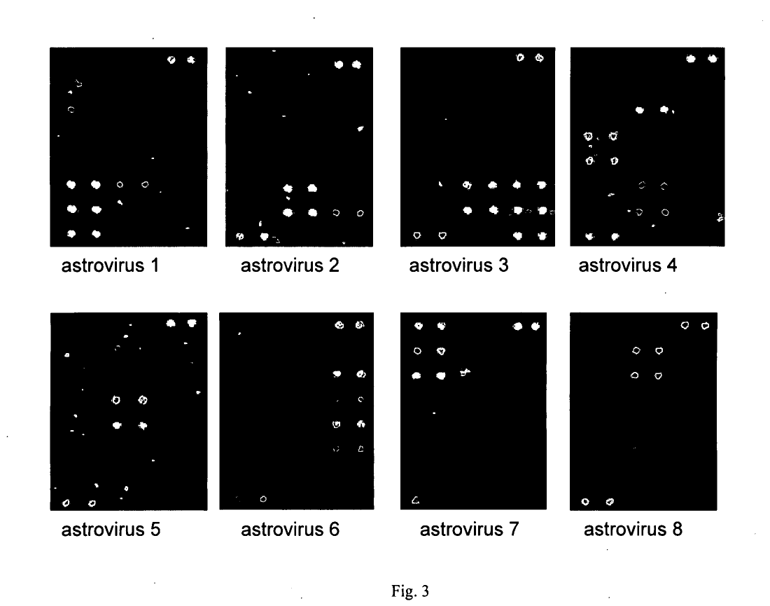

The methods and arrays of the present invention further embody assessing the specific gastrointestinal disease or condition associated with the astrovirus. In this embodiment, the probes of the present invention correlate directly to a particular disease or condition (e.g., gastrointestinal illness), as described further herein. Such a method involves determining the presence, absence, level or percentage of nucleic acid molecules in the sample that hybridize to one or more nucleic acid molecules of the present invention, and comparing or analyzing the presence, absence, level or percentage of the one or more complexes at the one or more time points. Absence is defined herein as the level of a hybrid complex that is below a detectable level or limit. Based on the hybridization that occurs between the probes of the present invention and those found in the sample, a determination or diagnosis of the disease or condition, or treatment thereof, can be made. Once the specific astrovirus serotype of a particular sample is identified, an individual can be better diagnosed and/or treated for associated diseases or condition. For example, FIG. 3 shows results from a sample having been infected with various astrovirus serotypes. The results of such a test help a physician or qualified person to properly diagnose the illness, which impacts the type of treatment provided to the patient. In yet another embodiment, hybridization of the probes of the present invention can directly correlate with the presence of the illness, disease or condition (e.g., a diagnosis). Such methods include determining the presence or absence of nucleic acid molecules that hybridize to the probes of the present invention, and then determining diseases associated with that pattern (presence and/or absence) of nucleic acid molecules in the sample.

Furthermore, the methods of the present invention include monitoring treatment of diseases. For example, the treatment for gastroenteritis can be monitored after the patient has received the proper treatment with antiviral medications, hydration, other medications that alleviate symptoms. Symptoms of gastroenteritis include diarrhea, headache, malaise, nausea, abdominal pain, and vomiting. As such, one can compare the results of a baseline determination, with one or more determinations made after treatment has begun. In one example, an absence of certain nucleic acid sequences from the sample that hybridize to nucleic acid sequences of the present invention indicates that the virus has passed. Assessing levels at various stages or time points prior to and/or during the course of treatment provides a physician with information to make better, more informed decisions regarding treatment.

In addition to using microarrays, assaying the nucleic acid molecules of the present invention can be conducted using several methods and in one embodiment includes a Southern blot. Briefly, blot techniques include immobilizing or attaching nucleic acid molecules to a solid support, and subjecting or contacting nucleic acid molecules obtained from a sample under conditions for hybridization. Methods for preparing the nucleic acid molecules from the sample are further described herein. In nucleic acid hybridization reactions, the conditions used to achieve a particular level of stringency are described herein and depend on the nature of the nucleic acids being hybridized. For example, the length (e.g., 18-24 mer), degree of complementarity, nucleotide sequence composition (e.g., GC v. AT content), and nucleic acid type (e.g., RNA v. DNA v. PNA) of the hybridizing regions of the nucleic acids can be considered in selecting hybridization conditions.

Also, amplification of polynucleotide sequence by, for example, the polymerase chain reaction (PCR) technique, further described herein, can serve the same purpose. By properly choosing the primers, one can obtain an amplified product of an expected size after a certain plurality of PCR cycles if the target sequence is present in the extracted sample containing nucleic acids or genetic material. This method offers sensitivity, since a 30-cycle reaction can generate an amplification on the order of 109.

The present invention includes methods of making an array. The method includes selecting a solid support, as described herein. In one embodiment, epoxide slides were used. The nucleic acid molecules shown in FIG. 1 can be synthesized by standard methods, and spotted onto the solid support, or they can be synthesized directly on the chip (in situ or in silico) through known processes. In one aspect, the nucleic acid molecules of the present invention can be grown on the solid support or integrated on the solid support using flow channels. Methods of forming high density arrays of oligonucleotides that are now known or developed in the future can be used to construct the array of the present invention, namely an array having the nucleic acid molecules described herein. In particular, arrays can be synthesized on a solid substrate by a variety of methods, including, but not limited to, light-directed chemical coupling, and mechanically directed coupling. See Pirrung et al., U.S. Pat. No. 5,143,854 (see also PCT Application No. WO 90/15070) and Fodor et al., PCT Publication Nos. WO 92/10092 and WO 93/09668 which disclose methods of forming vast arrays. See also, Fodor et al., Science, 251, 767-77 (1991). One example of synthesizing a polymer array includes the VLSIPSTM approach. Additionally, methods which can be used to generate an array of oligonucleotides on a single substrate can be used. For example, reagents are delivered to the substrate by either (1) flowing within a channel defined on predefined regions or (2) “spotting” on predefined regions. However, other approaches, as well as combinations of spotting and flowing, or other approaches can be employed.

The method further includes preparing the nucleic acid molecules for attachment to the solid support. Optionally, a spacer that provides a space between the support and the capture nucleotide sequences can be used to increase sensitivity of the array. A spacer that can be used with the present invention includes any molecular group that allows the nucleic acid molecule to remain off of or separated from the support. Another example of a spacer is a hexaethylene glycol derivative for the binding of small oligonucleotides upon a membrane. Patent publication No.: EP-0511559. In one embodiment of the invention, the nucleic acid probes of this invention comprise at least two parts, the specific probe, and the spacer/linker section. The specific probe portion comprises about 14-30 nucleic acids or nucleic acid mimetics (e.g., PNAs). The spacer/linker is comprised of anything that positions the specific probe away from the substrate and that adheres or attaches the specific probe to the substrate. Additionally, attachment to a gel can be done through either a direct covalent linkage to the acrylamide via the acrydite modification (Rehman et al. NAR(1999)vol27(2):649) or a covalent linkage to an “activated” acrylamide (NHS-ester, for example, CodeLink thin-film slides and Biocept gel pad slides) via an amine modification.

The nucleic acid molecules of the present invention can also be prepared to promote attachment to the solid support chosen, or to react with a coating placed on the support. The solid support can be coated to promote adherence to the support, and once the nucleic acid molecule is applied, in some cases ultraviolet irradiation allows for DNA fixation. For example, the nucleic acid molecules of the present invention or the solid support can be modified to react with substrates including amine groups, aldehydes or epoxies to promote attachment. As shown in the Exemplification, the 17 mer oligonucleotides were synthesized with I-linker modification and printed on the slides. Methods, now known or developed later, for promoting attachment of the nucleic acid to the solid support can be used.

The nucleic acid molecules of the present invention can be applied to the solid support with a spotter, a robotic machine that applies the droplets of the nucleic acid molecules of the present invention to a well or spot on the array. Many spotters used ink jet technology or the piezoelectric capillary effect to complete the grid of probe droplets. Spotting the nucleic acid molecules onto the solid support is often referred to as “printing.” The droplets of the nucleic acid molecules can be arranged in a desired format, so long as each sequence is bound to the surface in a different localized area. Multiple arrays can be placed on a single support, and the same array can be repeated more than once (e.g., between about 1 and 48 arrays). The number of arrays on the slide can be impacted by a combination of probe density and hybridization chamber “mask” size, or format. The hybridization chamber mask allows one to analyze multiple target samples on the same slide; each chamber creates its own physical separation. Currently, these mask options are 16-well from e.g., Grace BioLabs (described in obtaining the data described in the Exemplification). Others are also available from The Gel Company in a 24-well format and from Schott-Nexterion in a 16 and 48-well format. Additional formats known in the art and developed in the future can be used.

The present invention includes kits. Kits can include the array of the present invention, as described herein. Kits can also include reagents that are used to carry out hybridization. Examples of such regents include labeling reagents, primers that are specific to a conserved region of the astrovirus serotypes being assessed (labeled and/or unlabeled), buffers and washing solutions. Labeling reagents include labels, as described herein (e.g., fluorescent dyes, streptavidin conjugate, magnetic beads, dendrimers, radiolabels, enzymes, colorimetric labels, nanoparticles, and/or nanocrystals) including Cy3 and Cy5. The kit can also include software use to analyze the results, as described herein.

The present invention, in one embodiment, includes an isolated nucleic acid molecule having a nucleic acid sequence of any one of SEQ ID NOs:5-24; a nucleic acid sequence having between about 40% and about 100% of contiguous nucleotides of any one of SEQ ID NO: 5-24; any one of SEQ ID NOs:5-24; a nucleic acid sequence having between about 9 and about 25 contiguous nucleotides of any one of SEQ ID NO: 5-24; a sequences that hybridizes thereto; a reverse complement thereof, and any combination thereof. The present invention includes sequences as recited in FIG. 1.

As used herein, the terms “DNA molecule” or “nucleic acid molecule” include both sense and anti-sense strands, cDNA, complementary DNA, recombinant DNA, RNA, wholly or partially synthesized nucleic acid molecules, PNA and other synthetic DNA homologs. A nucleotide “variant” is a sequence that differs from the recited nucleotide sequence in having one or more nucleotide deletions, substitutions or additions so long as the molecules binds to the nucleic acid molecules of the present invention including its reverse complement. Such variant nucleotide sequences will generally hybridize to the recited nucleotide sequence under stringent conditions.

As used herein, an “isolated” gene or nucleotide sequence which is not flanked by nucleotide sequences which normally (e.g., in nature) flank the gene or nucleotide sequence (e.g., as in genomic sequences). Thus, an isolated gene or nucleotide sequence can include a nucleotide sequence which is designed, synthesized chemically or by recombinant means.

Also encompassed by the present invention are nucleic acid sequences, DNA or RNA, PNA or other DNA analogues, which are substantially complementary to the DNA sequences and which specifically hybridize with their DNA sequences under conditions of stringency known to those of skill in the art. As defined herein, substantially complementary means that the nucleic acid need not reflect the exact sequence of the sequences of the present invention, but must be sufficiently similar in sequence to permit hybridization with nucleic acid sequence of the present invention under high stringency conditions. For example, non-complementary bases can be interspersed in a nucleotide sequence, or the sequences can be longer or shorter than the nucleic acid sequence of the present invention, provided that the sequence has a sufficient number of bases complementary to the DNA of the serotype to be identified to allow hybridization therewith.

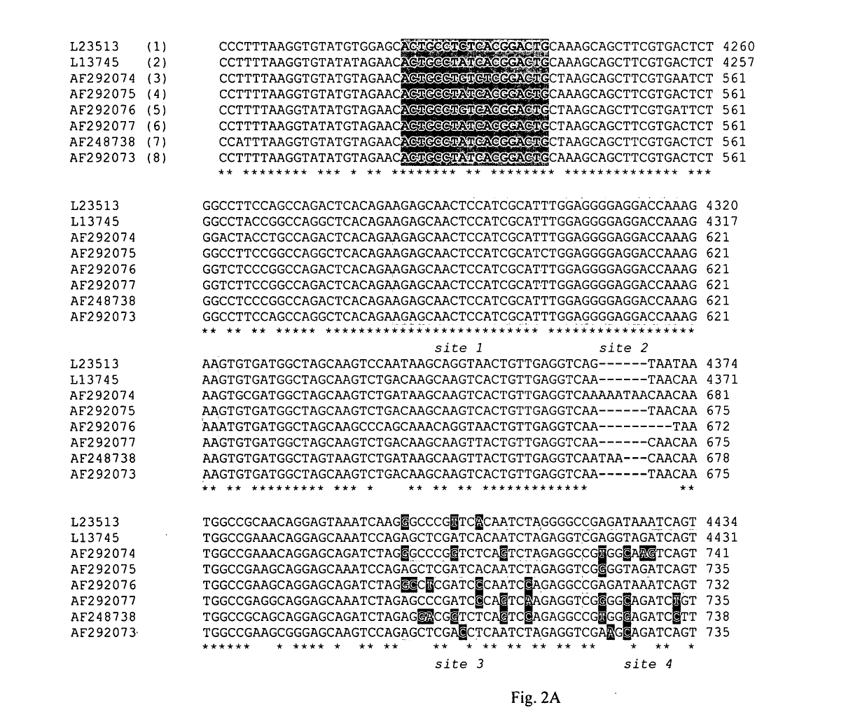

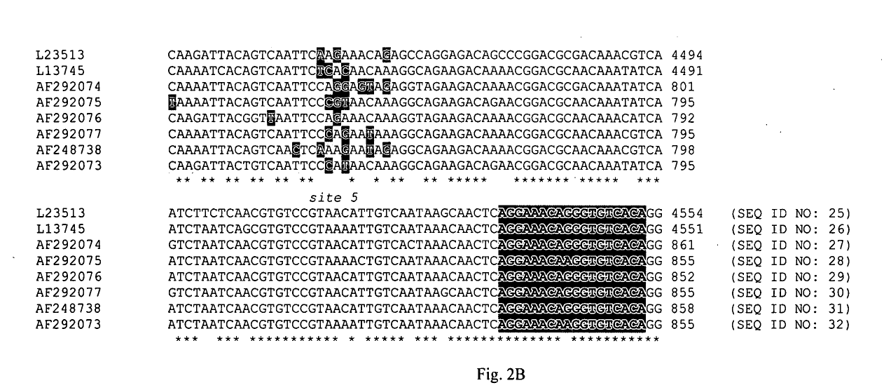

In another embodiment, the present invention includes molecules that contain at least about 9 to about 25 contiguous or tiled nucleotides or longer in length (e.g., 10, 11, 12, 13, 14, 15, 16, 17, 18, 19, 20, 21, 22, 23, or 24) of any nucleic acid molecules described herein, and preferably of SEQ ID NO: 5-24. “Tiled” probe designs are ones that use the sequences of the present invention but are just shifted 5′ or 3′ by 1 or more nucleotides. Alternatively, molecules of the present invention includes nucleic acid sequences having contiguous nucleotides of about 40% and about 100% of the length of any one of the sequences described herein, and preferably of SEQ ID NO: 5-24. The targets (e.g., SEQ ID NO: 25-32) provided herein can be used, but modified slightly by shifting the target in the astroviral serotype sequence by about 1 to about 12 nucleic acid bases in either direction (3′ or 5′). In such a case, an overlap the target sequence described herein occurs. Shifting the probe's target nucleic acid molecules by a few bases would allow one, in some cases, to still identify the particular serotype. When shifting of about 1 to about 10 bases of the 17 mer polynucleotide occurs, at least about 7 contiguous nucleotides of the sequences shown in FIG. 1 are used. When shifting of about 3 to about 5 bases of the 17 mer polynucleotide occurs, at least about 12 contiguous nucleotides of the sequences shown in FIG. 1 are used. Along the same lines, the nucleic acid molecules of the present invention can contain about 7 bases of the probes and up to about 12 bases of adjacent sequence from the astroviral serotype sequence, as provided in FIG. 2. Consequently, the nucleic acid molecules of the present invention can have about 30% or greater (about 40%, 50%, 60%, 70%, 75%, 80%, 85%, 90% or 95%) of contiguous or tiled nucleotides of the nucleic acid sequence described herein.

Similarly, the present invention includes nucleic acid probes that comprise the nucleic acid sequence of SEQ ID NO: 3-24 and/or is of sufficient length and complementarity to specifically hybridize to a nucleic acid sequence that identifies the corresponding serotype. The requirements of sufficient length and complementarity can be determined by one of skill in the art. Suitable hybridization conditions (e.g., high stringency conditions) are also described herein.

Specific hybridization can be detected under high stringency conditions. “Stringency conditions” for hybridization is a term of art which refers to the conditions of temperature and buffer concentration which permit and maintain hybridization of a particular nucleic acid to a second nucleic acid; the first nucleic acid may be perfectly complementary to the second, or the first and second may share some degree of complementarity which is less than perfect. For example, certain high stringency conditions can be used which distinguish perfectly complementary nucleic acids from those of less complementarity. “High stringency conditions” for nucleic acid hybridizations and subsequent washes are explained, e.g., on pages 2.10.1-2.10.16 and pages 6.3.1-6 in Current Protocols in Molecular Biology (Ausubel, et al., In: Current Protocols in Molecular Biology, John Wiley & Sons, (1998)). The exact conditions which determine the stringency of hybridization depend not only on ionic strength, temperature and the concentration of destabilizing agents such as formamide, but also on factors such as the length of the nucleic acid sequence, base composition, percent mismatch between hybridizing sequences and the frequency of occurrence of subsets of that sequence within other non-identical sequences. Thus, high stringency conditions can be determined empirically.

By varying hybridization conditions from a level of stringency at which no hybridization occurs to a level at which hybridization is first observed, conditions which will allow a given sequence to hybridize (e.g., selectively) with the most similar sequences in the sample can be determined. Exemplary conditions are described in the art (Krause, M. H., et al., 1991, Methods Enzymol. 200:546-556). Also, low and moderate stringency conditions for washes are described (Ausubel, et al., In: Current Protocols in Molecular Biology, John Wiley & Sons, (1998)). Washing is the step in which conditions are usually set so as to determine a minimum level of complementarity of the hybrids. Generally, starting from the lowest temperature at which only homologous hybridization occurs, each ° C. by which the final wash temperature is reduced (holding SSC concentration constant) allows an increase by 1% in the maximum extent of mismatching among the sequences that hybridize. Generally, doubling the concentration of SSC results in an increase in Tm of about 17° C. Using these guidelines, the washing temperature can be determined empirically for high stringency, depending on the level of the mismatch sought. In some embodiments, high stringency conditions include those in which nucleic acid with less than a few mismatches does not bind. Specific high stringency conditions used to carrying out the steps of the present invention are described in the Exemplification. High stringency conditions, using these guidelines, lie in a temperature range between about 40° C. and about 60° C., an SSC concentration range between about 1× and about 10× (e.g., about 2×), and a reaction time range of between about 30 seconds and about 36 hours.

### EXEMPLIFICATION

### Example 1

### MATERIALS AND METHODS

### Cell Culture and Virus Strains:

Viral isolates representative of the 8 known astrovirus serotypes were tested. Original seed viruses for astroviruses types 1-7 were obtained from a laboratory from Oxford, England. These were passed four times in Caco-2 cells for use as stock viruses. Caco-2 cells were obtained from the American Type Culture Collection, Manassas, Va. The cells were grown in D-MEM medium with 10% fetal bovine serum added. Additionally, a stool sample containing astrovirus type 8 was obtained from a laboratory from the University of Cambridge, Cambridge, U.K. This sample was used for the present study directly. For virus passage, cells were rinsed twice with serum-free D-MEM and inoculated with 100 μl of original seed or passaged virus stocks at 37° C. for one hour. The inoculum was removed and 1.0 ml of D-MEM containing 100 units penicillin, 100 μg streptomycin, 10 μg gentamicin, 1.0 μg amphotericin B, and 20 μg porcine trypsin 1:250 (Gibco BRL, Grand Island, N.Y.) per ml was added. The porcine trypsin used contained a minimum of 225 USP U/mg BAEE units of activity.

### Isolation of Viral RNA:

Viral RNA was purified from supernates of 10% fecal suspensions or cell cultures using Qiagen's QIAamp Viral RNA Mini Kit using the manufacturer's instructions.

### Design of Primers for RT-PCR:

ClustalW analysis of astrovirus ORF1b genomic sequences was used to produce an alignment for subsequent primer selection. Primers were selected using Premier Biosoft International's Primer Premier Version 5.0. A selection bias for low degeneracy and an optimum annealing temperature in the range of 49° C. to 52° C. was applied to the search.

### Microarray Probe Design:

The design concept was based on single nucleotide polymorphism (SNP) probe designs. SNP-probes typically contain a centrally located single point of variation that allows discrimination based on length of contiguous stretches of nucleotide identity, typically 25 nucleotides for perfect-match and 12 nucleotides for mismatch. Since the Astrovirus serotype sequences do not differ from each other at either the same point or a single point, any probe designed for one serotype sequence will have variable asymmetric points of variation relative to the different family member sequences. Consequently, short (17 nucleotide) oligonucleotide probes to three different regions within the astrovirus amplicon that contained different degrees of genetic variability among eight serological strains of the virus were designed (see FIG. 1). This allows for the potential discrimination between perfectly matched hybridization domains of 17-contiguous nucleotides and mismatched hybridization domains of shorter contiguous stretches, ranging from 2 to 11 nucleotides. In two cases, two astroviruses had the same sequence at the selected probe design sites (astrovirus 1 & 5 at site 4, and astrovirus 2 & 4 at site 3), so the respective oligonucleotide probes were excluded from this study.

### Microarray Production.

Short array probes (17-18mers, see FIG. 1) were each synthesized as a standard desalt purified oligonucleotide with a 5′ I-Linker modification (Integrated DNA Technologies, Coralville, Iowa). The oligonucleotide probe set was then printed at 40 μM in ESB, Epoxide Spotting Buffer, (Integrated DNA Technologies, Coralville, Iowa) on Corning Epoxide Slides using a BioRobotics MicroGrid 610 spotter equipped with Telechem 946 MP3 pins. Each oligonucleotide probe was spotted in duplicate per array with each slide containing 12 replicate arrays in a format compatible with the 16-chamber mask of the Grace Bio-Labs ProPlate™ Multi-Array Slide System. Printed slides were then treated for 1 hour in a humidity chamber with 84% humidity followed by 1 hour of drying in a desiccator. The slides were stored at room temperature until ready to hybridize.

### RT-PCR.

The astrovirus RT-PCR was performed using Qiagen's OneStep RT-PCR Kit. The sense primer (5′-ACTGCCTRTCWCGGACTG-3′) (SEQ ID NO: 1) and a modified Cy3-labeled antisense primer (5′-Cy3-T*G*T*GACACCYTGTTTCCT-3′) (* denotes position of phosphorothioate bonds) (SEQ ID NO: 2) were used at equimolar concentrations (final concentrations of 600 nM each in a 30 μl reaction volume). Following reverse transcription at 50° C. for 30 minutes, HotStarTaq DNA polymerase was activated by heating to 95° C. for 15 min. Ten cycles of denaturation, annealing, and extension at 94° C., 51° C., and 72° C., respectively were followed by an additional 10 cycles at 93° C., 52° C., and 72° C., and a final 20 cycles at 93° C., 53° C., and 72° C. Amplification ended with a 10 minute extension at 72° C.

Preparation of Single Stranded Cy3-Labeled Astrovirus Target cDNA.

Following RT-PCR with the labeled antisense primer, single Cy3-labeled targets were isolated using an a T7 exonuclease to preferentially degrade the strand complementary to the target strand (see U.S. application Ser. No. 12/190,446, filed Aug. 12, 2008 and U.S. Provisional No. 60/955,384, filed Aug. 12, 2007, which are incorporated by reference herein). Briefly, RT-PCR products were digested with a strand specific enzyme followed by column purification of the protected Cy3-labeled target strands using Promega ChipShot purification columns.

The strand specific digestion reaction consisted of 33 μl molecular biology grade water, 6 μl 10× digestion buffer, 1 μl enzyme (ten units), and 20 μl RT-PCR reaction product. Following a 2 hour incubation at room temperature, 6 μl sodium acetate (3M, pH 5.2) and 337.5 μl of binding solution were added to each 60 μl digestion reaction volume. Each mixture was gently mixed and applied to a Promega ChipShot purification column, incubated at room temperature for five minutes, and centrifuged at 10,000×g for 1 minute. The flow-through was discarded, and the column was washed with 500 μl 80% ethanol. Following centrifugation at 10,000×g for 1 minute, the flow-through was again discarded. The wash was repeated twice for a total of three washes. An additional centrifugation at 10,000×g for 1 minute was performed to remove residual ethanol.

For elution of target, the column was placed in a clean 2 ml collection tube, and Cy3-labeled ssDNA was eluted by adding 60 μl of elution buffer to each column. After two minutes incubation at room temperature, the column was centrifuged at 10,000×g for one minute. The eluted sample was dried down in a Speed-Vac. The dried Cy3-labeled ssDNA target was resuspended in 55 μl of 1× hybridization buffer, which consisted of 11 μl molecular biology grade water plus 44 μl 1.25×SNP Hybridization Buffer (Integrated DNA Technologies, Coralville, Iowa, as described in U.S. application Ser. No. 12/190,446, filed Aug. 12, 2008 and U.S. Provisional No. 60/955,384, filed Aug. 12, 2007, each of which is incorporated by reference in its entirety), to give a final concentration of 37.5 mM Tris pH 8, 3 mM EDTA, 0.25% Sarkosyl, 0.4 mg/mL Ovalbumin, 1 mM CTAB, 0.4 mg/mL Ficoll Type 400, 0.4 mg/mL PVP-360, 2.5M TMAC, 10% Formamide, 10 ug/mL Cot-1 DNA (1×SNP buffer).

### Microarray Hybridization

Prior to use, the slides were washed for 5 minutes with agitation using filtered, de-ionized water, rinsed for 1 minute in fresh water, and spun dry. The 16-chamber hybridization mask from the Grace Bio-Labs ProPlate™ Multi-Array Slide System was assembled onto the microarray slide. The resuspended Cy3-labeled ssDNA targets were heated for five minutes at 80° C. and pulse spun. 25 μl of each target/hybridization mix was applied to a single well of the 16-chamber mask on the array slide, covered with plastic film, and hybridized for 2 hours 15 minutes at 50° C. (in a humidity chamber in a water bath). The hybridization reaction was then removed by pipetting from each well. The hybridization mask was disassembled, and the slide immediately washed for 15 minutes in 200 ml of 1×SNP Wash Buffer 1 (2.5M TMAC and 0.2% Sarkosyl, as described in U.S. application Ser. No. 12/190,446, filed Aug. 12, 2008 and U.S. Provisional No. 60/955,384, filed Aug. 12, 2007) that had been preheated to 50° C. The wash buffer was maintained at 50° C. during the 15 minute wash. A second (200 ml 2×SSC buffer at room temperature) and third wash (200 ml 0.2×SSC buffer at room temp) were performed, and the slide was centrifuged at 1500×g to remove excess fluid.

### Scanning of the Microarray.

Hybridized slides were scanned using an Affymetrix 418 Scanner at an excitation wavelength of 532 nm and an emission wavelength of 570 nm. Laser power and gain for FIG. 3 were 80% and 50%, respectively.

### Sequencing of Astrovirus RT-PCR Products.

Astrovirus RT-PCR products were sequenced at the Tufts University Core Facility using an ABI 3100 automated DNA sequencer.

### Example 2

### Results

### Primers for RT-PCR.

The primers used for RT-PCR were characterized by low degeneracy; the antisense primer contained one variable nucleotide and the sense primer contained two variable nucleotides. RT-PCR products in relation to astrovirus sequences of eight serotypes are shown in FIG. 2. An asterisk under the sequences being compared indicates conserved nucleotides.

### Probes

The probes used for microarray analysis were 17 nucleotides in length (type-specific probes) or 18 nucleotides in length (conserved sequence probes). Their relative positions in the microarray are shown in FIG. 1, and their location relative to the amplified RT-PCR products are shown in FIG. 2.

### Hybridization

In FIG. 3, Cy3-labeled antisense targets obtained from amplification products of eight different serotypes of astrovirus were hybridized to the astrovirus microarray. Distinct patterns of hybridization were obtained for each of the eight viruses. For astrovirus 3, substantial hybridization was observed with the two astrovirus 2 probes. Level of binding to the astrovirus 2 probes suggested potential cross contamination with an astrovirus 2 sequence. A repeat RT-PCR and hybridization resulted in the expected binding pattern to astrovirus 1-specific probes.

The astrovirus 4 target bound to high levels to the astrovirus 8, site 5 probe. Based on the GenBank sequences used for probe design, the site 5 probes for astroviruses 4 and 8 should have differed by a single nucleotide (5′-CAATTCCCGTAACAAAG-3′ for astrovirus 4 versus 5′-CAATTCCCATAAACAAAG-3′ for astrovirus 8). Sequencing of the RT-PCR products for astroviruses 1 through 7 revealed that all sequences at sites 3, 4, and 5 were as expected except for site 5 of the astrovirus 4 isolate. This isolate was identical to astrovirus 8 as a result of a change of a single nucleotide from G to A. Additional astrovirus 4 sequences listed with GenBank reveal more variability at this site which will require the addition of more probe variants to the array. The astrovirus 8 labeled target bound as expected to the astrovirus 8, site 5 probe, demonstrating that a single nucleotide difference is sufficient to substantially impact target binding.

The relevant teachings of all the references, patents and/or patent applications cited herein are incorporated herein by reference in their entirety.

While this invention has been particularly shown and described with references to preferred embodiments thereof, it will be understood by those skilled in the art that various changes in form and details may be made therein without departing from the scope of the invention encompassed by the appended claims.

What is claimed is:

1. A method of detecting one or more human astrovirus serotypes in a group of astroviruses in a sample from an individual comprising:
(a) amplifying nucleic acid molecules of the sample with one or more primers specific to a conserved region of the astrovirus serotypes being assessed to obtain an amplified nucleic acid product;
(b) contacting the amplified nucleic acid product with one or more serotype specific probes having a nucleic acid sequence specific for a single astrovirus serotype in the group of astroviruses being assessed, the nucleic acid sequence having from about 9 o about 25 nucleic acid bases; and
(c) detecting hybridization between the amplified nucleic acid product the serotype specific probe, the presence of absence of hybridization indicating the presence or absence of one or more specific astrovirus serotypes in the sample.

2. The method of claim 1, wherein the amplification of the nucleic acid molecules is obtained using RT-PCR.

3. The method of claim 1, further including contacting the amplified nucleic acid product with one or more conserved sequence probes having a nucleic acid sequence that is specific for a conserved region shared by all astroviruses in the group of astroviruses being assessed.

4. The method of claim 3, the conserved sequence probes having a nucleic acid sequence of SEQ ID NO: 3 or 4 or a complement thereof, a nucleic acid sequence having from about 40% to about 100% of contiguous nucleotides of SEQ ID NO: 3 or 4 or a complement thereof, or a nucleic acid having from about 9 to about 25 contiguous nucleotides of SEQ ID NO: 3 or 4 or a complement thereof.

5. The method of claim 1, wherein the amplified nucleic acid product of step (a) comprises a detectable label.

6. The method of claim 1, wherein primers of step (a) comprise the nucleic acid sequences of SEQ ID NO: 1 or 2.

7. A method of detecting one or more human astroviruses in a sample from an individual, the method comprising:
(a) amplifying nucleic acid molecules of the sample with one or more primers specific to a conserved region of the astroviruses to produce an amplified nucleic acid product;
(b) contacting the amplified nucleic acid product under suitable hybridization conditions with one or more nucleic acid probe having a nucleic acid sequence of any one of SEQ ID NO: 5-24 or a complement thereof, a nucleic acid sequence having from about 40% to about 100% of contiguous nucleotides of any one of SEQ ID NO:5-24 or a complement thereof, or a nucleic acid having from about 9 to about 25 contiguous nucleotides of SEQ ID NO: 5-24 or a complement thereof; and.
(c) detecting the presence or absence of hybridization of the amplified nucleic acid product to the probe, the presence or absence of hybridization being indicative of the presence or absence of one or more serotype specific astroviruses.

8. The method of claim 7, further comprising contacting the amplified nucleic acid product with one or more conserved sequence probes having a nucleic acid sequence conserved sequence probes having a nucleic acid sequence of SEQ ID NO: 3 or 4 or a complement thereof, a nucleic acid sequence having from about 40% to about 100% of contiguous nucleotides of SEQ ID NO: 3 or 4 or a complement thereof, or a nucleic acid having from about 9 to about 25 contiguous nucleotides of SEQ ID NO: 3 or 4 or a complement thereof.

9. The method of claim 7, wherein the primers of step (a) comprise SEQ ID NO: 1 or 2.

10. The method of claim 7, wherein amplification of the nucleic acid molecules is obtained using RT-PCR.

11. The method of claim 7, wherein amplification of the nucleic acid molecules is obtained using asymmetric PCR.

12. A method for identifying an astrovirus serotype in a sample from an individual, the method comprises:
(a) reverse transcribing RNA from the sample to using one or more primers specific to a conserved region of the astrovirus serotypes to obtain DNA;
(b) optionally amplifying the DNA by PCR;
(c) labeling the DNA, prior to and/or during step (a) and/or step(b);
(d) contacting DNA of step (c) under conditions suitable for hybridization with one or more nucleic acid molecules having a nucleic acid sequence of any one of SEQ ID NO: 5-24 or complements thereof, or a nucleic acid sequence having between about 40% and about 100% of any contiguous nucleotides of SEQ ID NO: 5-24 or complements thereof, or a nucleic acid sequence having between about 9 and about 25 contiguous nucleotides of SEQ ID NO: 5-24 or complements thereof; and
(e) detecting the presence or absence of the hybridization, the presence of a complex indicates the presence of the serotype and the absence a complex indicates the absence of the serotype, wherein the serotype is astrovirus 1, astrovirus 2, astrovirus 3, astrovirus 4, astrovirus 5, astrovirus 6, astrovirus 7, or astrovirus 8.

13. A method of detecting one or more human astroviruses in a sample from an individual, the method comprises:
(a) isolating viral nucleic acid molecules from the sample;
(b) contacting one or more primers with the sample, the primers comprising nucleic acid sequence of SEQ ID NO: 1 or 2 and at least one primer comprising a tag, under conditions suitable for amplifying nucleic acid molecules of the sample, to obtain an amplified nucleic acid product having a labeled nucleic acid strand and an unlabeled nucleic acid strand;
(c) digesting the unlabeled nucleic acid strand to thereby obtained an amplified labeled single stranded nucleic acid product;
(d) contacting the amplified nucleic acid product under stringency conditions suitable for hybridization with one or more nucleic acid molecules having a nucleic acid sequence of any one of SEQ ID NO: 5-24 or a complement thereof, or a nucleic acid sequence having between about 40% and about 100% contiguous nucleotides of any one of SEQ ID NO: 5-24, or a complement thereof, or a nucleic acid sequence having between about 9 and about 25 contiguous nucleotides of SEQ ID NO: 5-24 or a complement thereof; and
(e) detecting hybridization of the amplified nucleic acid product to one or more nucleic acid molecules, the presence or absence of hybridization being indicative of the presence or absence of one or more specific astroviruses.

14. A method for diagnosing an individual having a disease or condition associated with an astrovirus, the method comprising:
determining the presence or absence of one or more nucleic acid molecules from a sample from the individual that hybridize to one or more nucleic acid molecules having a nucleic acid sequence of any one of SEQ ID NO: 5-24 or complements thereof, a nucleic acid sequence having between about 40% and about 100% of contiguous nucleotides of any of SEQ ID NO: 5-24 or complements thereof, a nucleic acid sequence having between about 9 and about 25 contiguous nucleotides or complements thereof, the presence or absence of one or more complexes indicates the presence or absence of the disease or condition.

15. A method for monitoring treatment or efficacy of therapy for an individual having a disease or condition associated with an astrovirus, the method comprising:
(a) determining the presence or absence of one or more nucleic acid molecules from at least two samples taken from the individual at different time points that hybridize to one or more nucleic acid molecules having a nucleic acid sequence one or more nucleic acid molecules having a nucleic acid sequence of any one of SEQ ID NO: 5-24 or complements thereof, a nucleic acid sequence having between about 40% and about 100% of contiguous nucleotides of any of SEQ ID NO: 5-24 or complements thereof, a nucleic acid sequence having between about 9 and about 25 contiguous nucleotides or complements thereof,
(b) comparing the hybridization of the nucleic acid molecules in the samples, wherein said comparison indicates the efficacy of therapy.

16. A device for the identification of one or more astrovirus serotypes, the device comprising a support having at least one array comprising a plurality of nucleic acid molecules deposited on the support in spatially distinct domains, the nucleic acid molecules having a nucleic acid sequence of any one of SEQ ID NO: 5-24 or complements thereof, a nucleic acid sequence having between about 40% and about 100% of contiguous nucleotides of any of SEQ ID NO: 5-24 or complements thereof, a nucleic acid sequence having between about 9 and about 25 contiguous nucleotides or complements thereof.

17. The device of claim 16, wherein the support comprises at least one of glass, silica chips, nylon membrane, polymer, plastic, ceramic, metal, and optical fiber.

18. The device of claim 17, wherein the solid support has from about one to about 48 different arrays.

19. The device of claim 18, wherein the device comprises the same array duplicated two or more times.

20. The device of claim 19, wherein more than one nucleic acid molecule is used to identify a serotype.

21. A kit comprising:
(a) the device of claim 20; and
(b) one or more reagents used for carrying out a nucleic acid hybridization assay.

22. The kit of claim 21, wherein the regents include at least one of a compound used to detect hybridization, unlabeled primers specific to a conserved region of human astrovirus serotypes, a labeled primers having a sequence specific to a conserved region of human astrovirus serotypes, washing solutions, hybridization buffers, amplification buffers, or exonuclease reaction buffers.

23. An isolated nucleic acid molecule from one or more astrovirus serotypes having a nucleic acid sequence of any one of SEQ ID NO: 5-24 or complements thereof, a nucleic acid sequence having between about 40% and about 100% of contiguous nucleotides of any of SEQ ID NO: 5-24 or complements thereof, a nucleic acid sequence having between about 9 and about 25 contiguous nucleotides or complements thereof.

24. The isolated nucleic acid molecule of claim 23, wherein the nucleic acid molecule is a DNA or RNA molecule.

25. The isolated acid molecules of claim 23, wherein the molecule is a probe that binds to a nucleic acid from an astrovirus serotype.

26. A method of making a device for the identification of an astrovirus serotype comprising:
depositing on a support an array of spatially arranged domains one or more nucleic acid molecules having a nucleic acid sequence of any one of SEQ ID NO: 5-24 or complements thereof, a nucleic acid sequence having between about 40% and about 100% of contiguous nucleotides of any of SEQ ID NO: 5-24 or complements thereof, a nucleic acid sequence having between about 9 and about 25 contiguous nucleotides or complements thereof.

27. The method of claim 26, wherein the nucleic acid molecules are deposited in a solution having a concentration of between about 1 μM and 200 μM.

28. The method of claim 26, wherein the support is a glass slide, and wherein from about 1 to about 16 arrays are deposited on the slide.

29. The method of claim 28, wherein the same array is duplicated 2 or more times.

30. The method of claim 26, further comprising synthesizing the nucleic acid molecule.

31. The method of claim 26, wherein the nucleic acid molecules are inserted or integrated within the solid support.


SEQUENCE LISTING (32 sequences)

SEQ ID NO: 1 - DNA, 18 bp, Artificial Sequence
  Oligonucleotide
actgcctrtcwcggactg

SEQ ID NO: 2 - DNA, 18 bp, Artificial Sequence
  Oligonucleotide
tgtgacaccytgtttcct

SEQ ID NO: 3 - DNA, 18 bp, Artificial Sequence
  Oligonucleotide
agagcaactccatcgcat

SEQ ID NO: 4 - DNA, 18 bp, Artificial Sequence
  Oligonucleotide
gaggggaggaccaaagaa

SEQ ID NO: 5 - DNA, 17 bp, Artificial Sequence
  Oligonucleotide
ggcccgttcacaatcta

SEQ ID NO: 6 - DNA, 17 bp, Artificial Sequence
  Oligonucleotide
ggcccggtctcagtcta

SEQ ID NO: 7 - DNA, 17 bp, Artificial Sequence
  Oligonucleotide
gcctcgatcccaatcca

SEQ ID NO: 8 - DNA, 17 bp, Artificial Sequence
  Oligonucleotide
agcccgatcccagtcaa

SEQ ID NO: 9 - DNA, 17 bp, Artificial Sequence
  Oligonucleotide
aggacggtctcagtcca

SEQ ID NO: 10 - DNA, 17 bp, Artificial Sequence
  Oligonucleotide
agctcgacctcaatcta

SEQ ID NO: 11 - DNA, 17 bp, Artificial Sequence
  Oligonucleotide
cgaggtagatcagtcaa

SEQ ID NO: 12 - DNA, 17 bp, Artificial Sequence
  Oligonucleotide
cgtggcaagtcagtcaa

SEQ ID NO: 13 - DNA, 17 bp, Artificial Sequence
  Oligonucleotide
cggggtagatcagttaa

SEQ ID NO: 14 - DNA, 17 bp, Artificial Sequence
  Oligonucleotide
cggggcagatctgtcaa

SEQ ID NO: 15 - DNA, 17 bp, Artificial Sequence
  Oligonucleotide
cgaagcagatcagtcaa

SEQ ID NO: 16 - DNA, 17 bp, Artificial Sequence
  Oligonucleotide
cgaagcagatcagtcaa

SEQ ID NO: 17 - DNA, 17 bp, Artificial Sequence
  Oligonucleotide
caattcaagaaacagag

SEQ ID NO: 18 - DNA, 17 bp, Artificial Sequence
  Oligonucleotide
caattctcacaacaaag

SEQ ID NO: 19 - DNA, 17 bp, Artificial Sequence
  Oligonucleotide
caattccaggagtagag

SEQ ID NO: 20 - DNA, 17 bp, Artificial Sequence
  Oligonucleotide
caattcccgtaacaaag

SEQ ID NO: 21 - DNA, 17 bp, Artificial Sequence
  Oligonucleotide
caattcccagaataaag

SEQ ID NO: 22 - DNA, 17 bp, Artificial Sequence
  Oligonucleotide
caattcccagaataaag

SEQ ID NO: 23 - DNA, 17 bp, Artificial Sequence
  Oligonucleotide
caactcaaagaatagag

SEQ ID NO: 24 - DNA, 17 bp, Artificial Sequence
  Oligonucleotide
caattcccataacaaag

SEQ ID NO: 25 - DNA, 354 bp, Artificial Sequence
  Oligonucleotide
ccctttaaggtgtatgtggagcactgcctctcacggactgcaaagcagcttcgtgactctggccttccagccagactcacagaagagcaactccatcgcatttggaggggaggaccaaagaagtgtgatggctagcaagtccaataagcaggtaactgttgaggtcagtaataatggccgcaacaggagtaaatcaagggcccgttcacaatctaggggccgagataaatcagtcaagattacagtcaattcaagaaacagagccaggagacagcccggacgcgacaaacgtcaatcttctcaacgtgtccgtaacattgtcaataagcaactcaggaaacagggtgtcacagg

SEQ ID NO: 26 - DNA, 354 bp, Artificial Sequence
  Oligonucleotide
ccttttaaggtgtatatagaacactgcctatcacggactgcaaagcagcttcgtgactctggcctaccggccaggctcacagaagagcaactccatcgcatttggaggggaggaccaaagaagtgtgatggctagcaagtctgacaagcaagtcactgttgaggtcaataacaatggccgaaacaggagcaaatccagagctcgatcacaatctagaggtcgaggtagatcagtcaaaatcacagtcaattctcacaacaaaggcagaagacaaaacggacgcaacaaatatcaatctaatcagcgtgtccgtaaaattgtcaataaacaactcaggaaacagggtgtcacagg

SEQ ID NO: 27 - DNA, 360 bp, Artificial Sequence
  Oligonucleotide
ccttttaaggtgtatgtagaacactgcctgtctcggactgctaagcagcttcgtgaatctggactacctgccagactcacagaagagcaactccatcgcatttggaggggaggaccaaagaagtgcgatggctagcaagtctgataagcaagtcactgttgaggtcaaaaataacaacaatggccgaaacaggagcagatctagggcccggtctcagtctagaggccgtggcaagtcagtcaaaattacagtcaattccaggagtagaggtagaagacaaaacggacgcgacaaatatcagtctaatcaacgtgtccgtaacattgtcactaaacaactcaggaaacagggtgtcacagg

SEQ ID NO: 28 - DNA, 354 bp, Artificial Sequence
  Oligonucleotide
ccttttaaggtgtatgtagaacactgcctatcacggactgcaaagcagcttcgtgactctggccttccggccaggctcacagaagagcaactccatcgcatctggaggggaggaccaaagaagtgtgatggctagcaagtctgacaagcaagtcactgttgaggtcaataacaatggccgaagcaggagcaaatccagagctcgatcacaatctagaggtcggggtagatcagttaaaattacagtcaattcccgtaacaaaggcagaagacagaacggacgcaacaaatatcaatctaatcaacgtgtccgtaaaactgtcaataaacaactcaggaaacaaggtgtcacagg

SEQ ID NO: 29 - DNA, 351 bp, Artificial Sequence
  Oligonucleotide
ccttttaaggtatatgtagaacactgcctgtcacggactgctaagcagcttcgtgattctggtctcccggccagactcacagaagagcaactccatcgcatttggaggggaggaccaaagaaatgtgatggctagcaagcccagcaaacaggtaactgttgaggtcaataatggccgaagcaggagcagatctaggcctcgatcccaatccagaggccgagataaatcagtcaagattacggttaattccagaaacaaaggtagaagacaaaacggacgcaacaaacatcaatctaatcaacgtgtccgtaacattgtcaataaacaactcaggaaacagggtgtcacagg

SEQ ID NO: 30 - DNA, 354 bp, Artificial Sequence
  Oligonucleotide
ccttttaaggtatatgtagaacactgcctatcacggactgctaagcagcttcgtgactctggtcttccggccagactcacagaagagcaactccatcgcatttggaggggaggaccaaagaagtgtgatggctagcaagtctgacaagcaagttactgttgaggtcaacaacaatggccgaggcaggagcaaatctagagcccgatcccagtcaagaggtcggggcagatctgtcaaaattacagtcaattcccagaataaaggcagaagacaaaacggacgcaacaaacgtcagtctaatcaacgtgtccgtaacattgtcaataagcaactcaggaaacagggtgtcacagg

SEQ ID NO: 31 - DNA, 357 bp, Artificial Sequence
  Oligonucleotide
ccatttaaggtgtatgtagaacactgcctatcacggactgctaagcagcttcgtgactctggcctcccggccagactcacagaagagcaactccatcgcatttggaggggaggaccaaagaagtgtgatggctagtaagtctgataagcaagttactgttgaggtcaataacaacaatggccgcagcaggagcagatctagaggacggtctcagtccagaggccgtgggagatccttcaaaattacagtcaactcaaagaatagaggcagaagacaaaacggacgcaacaaacgtcaatctaatcaacgtgtccgtaacattgtcaataaacaactcaggaaacagggtgtcacagg

SEQ ID NO: 32 - DNA, 354 bp, Artificial Sequence
  Oligonucleotide
ccttttaaggtatatgtagaacactgcctatcacggactgcaaagcagcttcgtgactctggccttccagccaggctcacagaagagcaactccatcgcatttggaggggaggaccaaagaagtgtgatggctagcaagtctgacaagcaagtcactgttgaggtcaataacaatggccgaagcgggagcaagtccagagctcgacctcaatctagaggtcgaagcagatcagtcaagattactgtcaattcccataacaaaggcagaagacagaacggacgcaacaaatatcaatctaatcaacgtgtccgtaaaattgtcaataaacaactcaggaaacaaggtgtcacagg